(19) United States
(12) Patent Application Publication
(10) Pub. No.: US 2007/0286808 A1
(43) Pub. Date: Dec. 13, 2007

(54) METHOD FOR DISPLAY PRESENTATION OF LYMPH NODES

(76) Inventor: Ali-Nejat Bengi, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/755,132

(22) Filed: May 30, 2007

(30) Foreign Application Priority Data
Jun. 8, 2006 (DE) 10 2006 026 723.0

(51) Int. Cl. A61K 49/10; A61B 5/055

(52) U.S. Cl. 424/9.32; 600/410

(57) ABSTRACT

In a method for display presentation of lymph nodes of an examination person by a magnetic resonance imaging, a contrast agent with super-paramagnetic iron oxide particles is injected into the examination person, an MR imaging sequence for phase-sensitive acquisition of the lymph nodes of the examination person is implemented, at least one phase image and one magnitude image of the acquired MR data are computationally generated, a mask is generated from the phase image, the magnitude image is filtered with the mask, and the filtered magnitude image is displayed.

### BACKGROUND OF THE INVENTION

1. Field of the Invention

The present invention concerns a method for display presentation of lymph nodes of an examination person by magnetic resonance tomography.

2. Description of the Prior Art

The detection of metastases in lymph nodes plays an important role in the diagnosis of cancer illnesses since the chemotherapy, radiation therapy and/or the surgical procedure must be established dependent on the pathology of the lymph nodes.

Lymph examinations are typically implemented by means of lymph angiography, computed tomography, magnetic resonance tomography, or PET. For examinations by means of magnetic resonance tomography, the exam is for the most part based on use of the size of the lymph nodes as a criterion for the diagnosis. The precision of this method is, however, not very high.

Furthermore, ultra-small, super-paramagnetic iron oxide particles have been developed as a contrast agent (for example Ferumoxtran-10) that are injected into an examination subject and can be used as a contrast agent in an intravenous lymph angiography. As is explained in detail later, this contrast agent is absorbed by the macrophages in the lymph nodes. In magnetic resonance examinations this paramagnetic portion in the iron oxide particles effects an attenuation of the signal intensity due to the susceptibility effects of the iron oxide, causing the T2* time to be shortened. This change of the MR signal is conventionally shown with high-resolution MR imaging sequences; these imaging sequences must be sensitive to the T2* time. An example of such super-paramagnetic iron oxide particles for representation of the lymph nodes is described in Radiology 2002/222, pages 239-244 by Martin G. Mack et al. Small changes in lymph nodes, however, cannot be detected with conventional T2*-weighted gradient echo sequences.

Furthermore, an acquisition technique that is used under the name SWI (Susceptibility Weighted Imaging) is known from U.S. Pat. No. 6,501,272. In addition to the MR magnitude image, the phase information in the MR image is used in this imaging technique in order to show susceptibility differences between oxygen-poor and oxygen-rich blood.

### SUMMARY OF THE INVENTION

An object of the present invention is to further improve the representation of lymph nodes in magnetic resonance tomography, in particular the detection of metastases in the lymph nodes.

This object is achieved in accordance with the invention by a method for display presentation of lymph nodes of an examination person by magnetic resonance imaging, wherein a contrast agent with very small super-paramagnetic iron oxide particles is injected into the examined subject, and after a predetermined wait time, a phase-sensitive imaging sequence for imaging the lymph nodes of the examination person is implemented. A phase image and a magnitude image are subsequently calculated from the acquired MR data. A mask can then be generated from the phase image, which mask is subsequently used for filtering of the magnitude image. Finally, the magnitude image filtered with the phase mask can be shown. The sensitivity in the detection of metastases in lymph nodes can be increased via the use of the phase information as additional information. The injected contrast agent is absorbed by the macrophages in the lymph nodes. In the case of a metastasis, this exhibits a lower absorption of the contrast agent. Since the contrast agent attenuates the signal intensity and since this signal attenuation is reduced in metastases, the differentiation between healthy tissue in lymph nodes and metastasized tissue can be improved with the use of the different phase information.

According to a preferred embodiment, the MR data are acquired using a gradient echo sequence. This gradient echo sequence can be a two-dimensional gradient echo sequence or a three-dimensional gradient echo sequence. Naturally, any other imaging sequence can also be used as long as it is ensured that the acquired MR data are sensitive to magnetic field changes. For example, echoplanar imaging sequences can also be used.

Furthermore, a flow compensation and/or a movement compensation can be implemented in the imaging. A flow compensation can be executed in order to minimize the influence of blood flow. A movement compensation can be necessary in order, for example, to take breathing movements of the examination person into account. Methods known as navigator techniques that detect the movement of the diaphragm can be used for detection of the breathing movement, with the signal acquisition of the MR signal being triggered at predetermined breathing positions.

Furthermore, the calculated phase image can be subjected to high-pass filtering in order to reduce inhomogeneities of the magnetic field and image artifacts at air-tissue transitions. Regions with increased signal intensity in the lymph nodes can advantageously be sought in the filtered magnitude image since regions with increased signal intensity in the lymph nodes indicate metastases.

After injection of the contrast agent a wait must occur until the contrast agent travels out from the bloodstream across the interstice into the lymph nodes. A time span between 20 and 30 hours preferably will occur between the contrast agent injection and the MR acquisitions. Given too short a wait time, the contrast agent has not yet arrived in the lymph nodes; given too long a wait time, the contrast agent is no longer contained in sufficient concentration in the lymph nodes.

In order to improve the presentation of metastases in lymph nodes, for the generation of the phase filter in the phase images a comparison is made between the signal intensity in image regions with enriched iron oxide particles and image regions without iron oxide particles.

### DESCRIPTION OF THE DRAWINGS

FIG. 1 schematically shows the path of the contrast agent from the bloodstream into the lymph channels in illustration (a), and shows a lymph node with a metastasis in illustration (b), and shows an enlarged section of the lymph node of illustration (b) in illustration (c).

FIG. 2 is a flow chart of the basic steps for improved presentation of metastases in an MR image in accordance with the invention.

### DESCRIPTION OF THE PREFERRED EMBODIMENTS

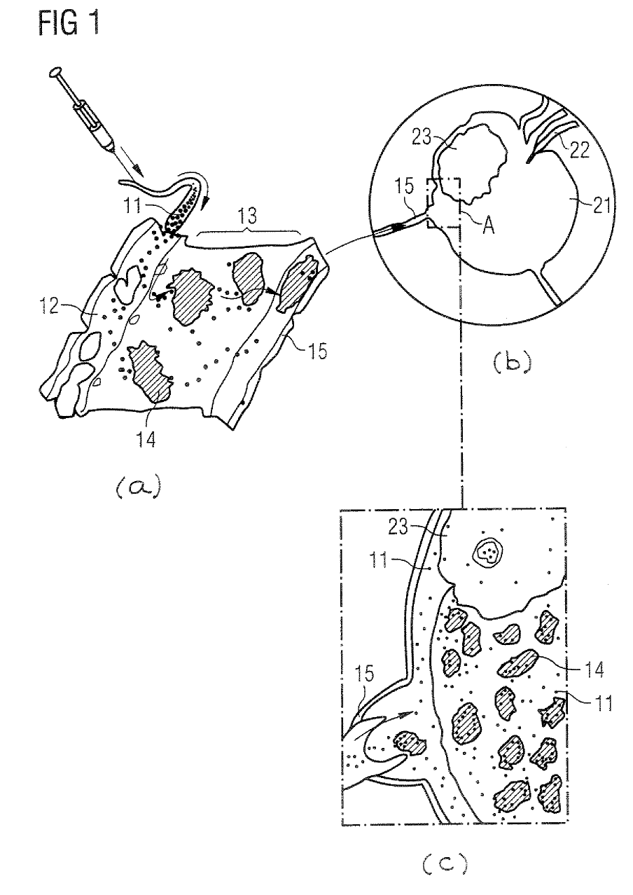

Illustration (a) in FIG. 1 shows an example of how the intravenously-injected contrast agent propagates in the body. The injected contrast agent has very small super-paramagnetic iron oxide particles 11 that are injected into the blood vessel 12. These very small super-paramagnetic iron oxide particles traverse the blood vessel 12 and travel through the vessel wall into the interstice 13 where macrophages 14 are found. The iron particles 11 are transported to the lymph channels 15 in this interstitial space. The iron oxide particles are transported by the lymph stream 15 to the lymph node 21 that is shown in illustration (b). The lymph node has incoming lymph channels 15 as well as continuative channels 22. A metastasis 23 is exemplarily shown in the lymph node.

The section A in illustration (b) is shown enlarged in illustration (c), whereby this section presents a metastasized tissue portion and a healthy tissue portion. As can be seen in illustration (c), the healthy tissue portion exhibits a number of macrophages 14 and iron particles 11 that are enclosed in the macrophages. Fewer iron particles 11 are contained in the tissue of the metastasis 23. The super-paramagnetic iron oxide particles alter the magnetic field and therewith the signal in the MR acquisition. These different magnetic fields lead to a different phase position of the nuclear spins in tissue with and without iron oxide particles. When the magnetic field change is ΔB, the phase given acquisition of a phase-sensitive acquisition sequence changes by Δφ=γΔBTE, whereby γ is the gyromagnetic ratio and TE is the echo time of the imaging sequence. If a pixel now contains tissue with and without iron particles, the total signal intensity is composed of

Iₜₒₜₐₗ=I_{background}+Iᵢᵣₒₙ exp(i-γΔBTE)

This means that the phase in the healthy tissue changes relative to that in metastasized tissue. The signal difference between healthy tissue and metastasis tissue is oscillating.

In order to now make the phase difference optimally large between metastasis tissue and non-metastasized tissue, the echo time must be appropriately selected so that the iron oxide particle-comprising tissue can be differentiated relative to tissue that does not comprise iron oxide particles. An echo time between 35 and 50 ms can be exemplarily adopted.

Naturally, the echo time depends on the basic magnetic field strength and on other factors and must be adapted to the remaining circumstances of the examination.

The magnitude signal intensity of image points that exhibit iron oxide-containing particles is less than the signal intensity of tissue without iron oxide particles. In the present case the signal intensity of tissue with iron oxide particles can now be determined. This is the reference phase (φ0. The phase of the tissue of the acquired lymph nodes can now be compared with the reference phase. For example, the phase filter can then be selected such that all phase positions within a range around the reference angle are populated with a value between 0 and 1 while phase positions outside of a predetermined range receive the value 1 or greater than 1. Other embodiments of the phase filter are also possible. More precise information with regard to the phase filter can be found in U.S. Pat. No. 6,501,272. Overalls the phase filter must be selected such that the signal intensity reduction in the healthy tissue parts is reduced relative to tissue portions with metastases, upon the subsequent multiplication with the magnitude image.

The total signal information then results from multiplications of the original signal intensity magnitude image with the modified phase image in which the phase position was changed as mentioned above.

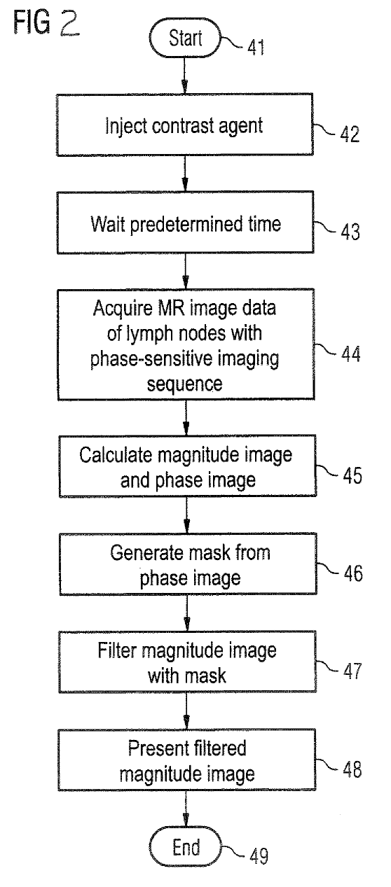

The steps that can be implemented for detection of slight changes of the lymph nodes are shown in summary in FIG. 2. After the start of the method in step 41 the contrast agent with the very small super-paramagnetic iron oxide particles is injected in step 42. After injection of the contrast agent a predetermined time must subsequently be waited until the contrast agent accumulates in the lymph nodes (step 43). This time span can be approximately 24 hours, but time spans of 20 or up to 35 hours are also possible. After this time span a phase-sensitive imaging sequence of the lymph nodes is implemented in a step 44. The imaging sequence can be a two-dimensional or three-dimensional gradient echo sequence. After acquisition of the MR data in step 44 the magnitude image and the phase image for each MR image are calculated in step 45. A mask is subsequently generated from the phase image in step 46. For example, the phase position of contrast agent-enriched tissue can hereby be determined and the phase position can be used as a reference phase, whereby the phase information of the remaining tissue is compared with the phase information of the reference tissue. In order to now amplify the signal intensity reduction in the healthy tissue relative to metastasized tissue, for example phase positions that lie within a specific range in proximity to the reference phase can be populated with values between 0 and 1 while phase positions that lie outside of a specific range of the reference phase can be populated with values greater than 1. Other types of processing of the phase image for generation of the phase filter are also possible as long as it is ensured that the phase image is processed such that the signal intensity attenuation in the magnitude image is increased in non-metastasized tissue relative to metastasis tissue. Finally, in step 47 the magnitude image is filtered (i.e. is multiplied) with the phase filter calculated from the phases, with the signal differences between healthy and metastasis tissue being amplified by the mask. The filtered magnitude image is subsequently shown in image 48 and smaller metastases in lymph nodes can now also be better identified in this filtered magnitude image, which was not possible with conventional three-dimensional gradient echo sequences without taking the phase position into account. The method ends in step 49.

In summary, the method described herein enables the detection of small metastases and very small lymph nodes by magnetic resonance tomography.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for display presentation of lymph nodes of an examination subject by magnetic resonance imaging, comprising the steps of:
injecting an examination with contrast agent comprising super-paramagnetic iron oxide particles;
acquiring magnetic resonance imaging data from a region of the examination subject containing lymph nodes, after injecting said contrast agent, by implementing a phase-sensitive magnetic resonance imaging sequence;
automatically computationally generating at least one phase image and at least one magnitude image of said lymph nodes from said magnetic resonance data;
automatically electronically generating a mask from said phase image;
automatically electronically filtering said magnitude image with said mask, to obtain a filtered magnitude; and
displaying said filtered magnitude image.

2. A method as claimed in claim 1 comprising implementing a gradient echo sequence as said imaging sequence.

3. A method as claimed in claim 1 comprising implementing a sequence with flow compensation as said imaging sequence.

4. A method as claimed in claim 1 comprising automatically electronically high-pass filtering said phase image before generating said mask from said phase image.

5. A method as claimed in claim 1 wherein said filtered magnitude image exhibits a signal intensity, and comprising automatically electronically analyzing said signal intensity of said magnitude image to identify portions of said filtered magnitude image with increased signal intensity in said lymph nodes as indicative of metastases.

6. A method as claimed in claim 1 comprising acquiring said magnetic resonance data of said lymph nodes in a time span between twelve and thirty-six hours following injection of said contrast agent.

7. A method as claimed in claim 1 comprising identifying a phase position of tissue containing said iron oxide particles in said region, and determining a phase position of remaining tissue in said region, and using said phase position of said remaining tissue in said region as a reference phase position for comparison with said phase position of said tissue containing iron oxide particles.

8. A method as claimed in claim 7 comprising generating said mask from said phase image by amplifying signal intensity attenuation in tissue in said region without metastases relative to tissue in said region with metastases.

9. A method as claimed in claim 7 comprising setting phase angles in said phase image that are within a predetermined range of said reference phase position to a value less than or equal to one, and setting phase angles outside of said predetermined region of said reference phase position to a value greater than or equal to one.

10. A method as claimed in claim 7 comprising setting phase angles in said phase image that are within a predetermined range of said reference phase position to a value less than or equal to one, and setting phase angles outside of said predetermined region of said reference phase position to a value greater than or equal to one.
